(19) United States
(12) Patent Application Publication
(10) Pub. No.: US 2011/0004321 A1
(43) Pub. Date: Jan. 6, 2011

(54) Interface method between an operator and a multiarticular robotic and/or prosthetic device for moving the same

(76) Inventor: Emanuele Lindo Secco, Romano D'Ezzelino VI (IT)

Correspondence Address: JACOBSON HOLMAN PLLC, 400 SEVENTH STREET N.W., SUITE 600, WASHINGTON, DC, 20004, US

(21) Appl. No.: 12/461,458

(22) Filed: Aug. 12, 2009

(30) Foreign Application Priority Data
Jul. 6, 2009 (IT) MI2009A 001185

(51) Int. Cl. A61F 2/48; G06F 19/00

(52) U.S. Cl. 623/24; 700/245

(57) ABSTRACT

The present invention refers to an interface method between an operator and a multiarticular robotic and/or prosthetic device for moving the same that is characterised in that it comprises the steps that consist of receiving (110) as an input a plurality of control signals whose number is less than a number of signals required by a plurality of actuators (12) used for moving a plurality of movable members (11, 13) of the multiarticular robotic and/or prosthetic device (10) and converting the plurality of input control signals into a number of output signals corresponding to the number of signals required by the actuators (12) by means of a plurality of principal components which characterize the movements of the device (10) within a moving space.

The present invention refers to an interface method between an operator and a multiarticular robotic and/or prosthetic device for moving the same.

In general, in the field of robotics, and in particular in the field of industrial robotics, there has been wide use of the so-called robotic arm or manipulator robot built to imitate the human arm, but often equipped with even more degrees of freedom.

Such anthropomorphic robots are widely made and used for example in assembly chains, allowing companies to substantially reduce costs as well as accelerating and improving production, but also replacing operators when hostile working conditions make human intervention difficult, like for example for repairs and/or maintenance carried out in space where there is no gravity or oxygen, or else in other dangerous or hard-to-reach environments for a person.

In the use of assembly chains, where the operations to carry out are clearly defined and are repeated, the driving of such robotic devices can take place in an automated and controlled manner only by electronic processors.

Otherwise, in more complex and variable operations it is necessary to use a human control made through interfacing methods between machine and man.

Such interfacing methods are generally very complex and require long training time for the operator in order to be able to accurately move a multiarticular robotic device having a large number of actuators used to manage the same number or more degrees of freedom.

Moreover, an operator is not always able to provide a number of input signals equal to the control signals required by the actuators used to manage the degrees of freedom offered by the robotic device to be driven.

For example, in the field of prosthetic devices in which it is attempted to recreate the functionality offered by the amputated limb, in general it is only possible to easily access a few electromyographic signals detected by muscle contractions that are currently insufficient to drive complex prosthetic devices having an amount of actuators such as to drive a number of degrees of freedom comparable to that of the limb that they replace.

As an alternative to electromyographic signals, it is possible to use a plurality of cortical signals to drive the different degrees of freedom offered by the prosthesis. However, this solution is too invasive, obtaining only poor adaptation and acceptance results by the patient.

Basically, the only approaches and methods for interfacing between a patient and a multiarticular prosthesis that have been proposed to date are invasive, costly and complex.

The purpose of the present invention is to avoid the aforementioned drawbacks and in particular to devise an interface method between an operator and a multiarticular robotic and/or prosthetic device for moving the same that simplifies the control in terms of a reduction in the inputs necessary to control the device with respect to the number of signals required by the actuators used to manage the degrees of freedom that it has.

Another purpose of the present invention is to provide an interface method between an operator and a multiarticular robotic and/or prosthetic device for moving the same that allows the operator to quickly learn how to manoeuvre the device driven.

A further purpose of the present invention is to make an interface method between an operator and a multiarticular robotic and/or prosthetic device for moving the same that, in patients with amputation trauma, allows an almost complete return to dexterity and a fast rehabilitation process.

These and other purposes according to the present invention are accomplished by making an interface method between an operator and a multiarticular robotic and/or prosthetic device for moving the same as outlined in claim 1.

Further characteristics of the interface method between an operator and a multiarticular robotic and/or prosthetic device for moving the same are the object of the dependent claims.

The characteristics and advantages of an interface method between an operator and a multiarticular robotic and/or prosthetic device for moving the same according to the present invention shall become clearer from the following description, given as a non-limiting example, referring to the attached schematic drawings, in which:

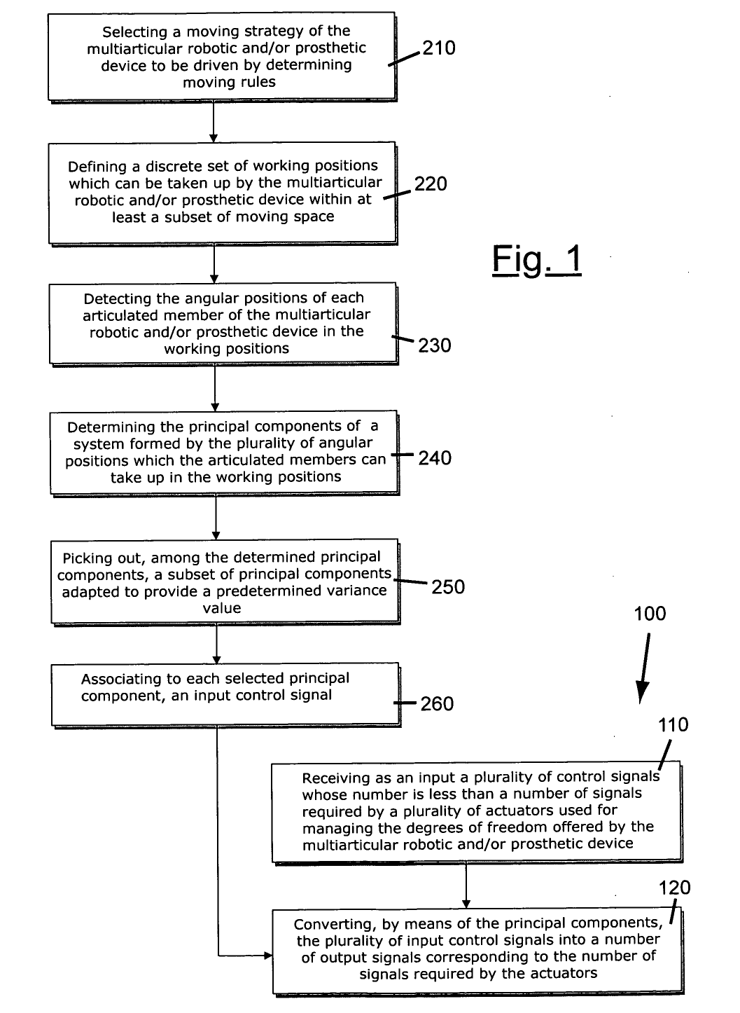

FIG. 1 is a block diagram of the interface method between an operator and a multiarticular robotic and/or prosthetic device for moving the same according to the present invention;

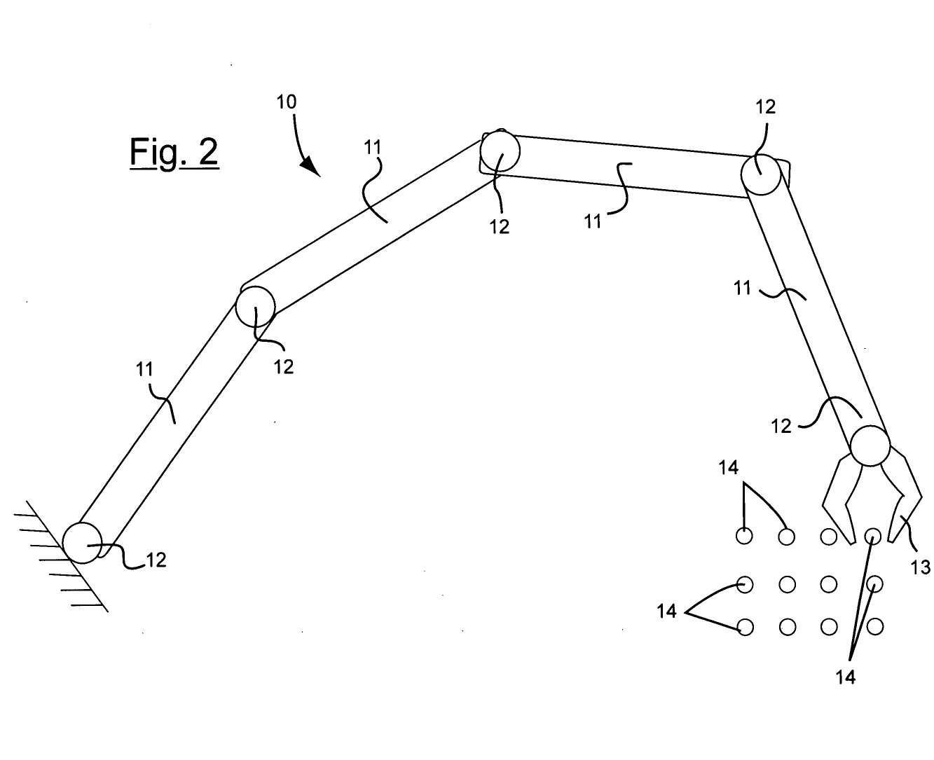

FIG. 2 is a schematic view of a multiarticular robotic device able to be driven through the method according to the present invention.

With reference to the figures, an interface method between an operator and a multiarticular robotic and/or prosthetic device for moving the same is illustrated, wholly indicated with 100.

Basically, according to the present invention, as an input to the driving interface of a multiarticular robotic and/or prosthetic device 10 a plurality of control signals is supplied (step 110) in lesser number with respect to the number of signals required by the set of actuators used to manage the degrees of freedom present in such a device 10 given by the possibility of moving a plurality of mobile members 11,13 of the same 10.

The input signals are therefore converted (step 120) into a number of output signals corresponding to the number of actuators present in the device 10 driven by means of principal components of the system equal to the number of input signals, where the principal components of the system are for example determined according to the following.

Firstly (step 210) a moving strategy of the multiarticular robotic and/or prosthetic device 10 to be driven by defining moving rules is selected.

Then, defining (step 220) a discrete set of working positions within a limited moving space which can be taken up by the multiarticular robotic and/or prosthetic device 10 to be driven that respects the defined moving strategy.

In practice, a plurality of postures and/or movements of the multiarticular robotic and/or prosthetic device 10 is determined through the experimental data acquisition or the determination of a model.

In a third step 230, for each working position the angular positions of the individual articulated members 11 that make up the multiarticular device 10 are detected.

Depending on the system thus discretised and described, the principal components of the system are determined (step 240).

Principal component analysis (also known as Karhunen-Loève transform or Hotelling transform) is a technique for simplifying data that in this case has the purpose of reducing the number of variables representing as many control inputs of the system into a number of latent variables.

The principal components of the system can be obtained by means of known methods currently able to be implemented in computing environments for an electronic computer available on the market, like for example MATLAB.

Then (step 250), among the principal components determined, a subset of principal components to be used in controlling the multiarticular robotic and/or prosthetic device 10 is picked out depending on the variance that is intended to be achieved.

In general, for systems having 15-20 degrees of freedom, with the two principal components that offer greatest variance, it is possible to achieve variance values equal to 80%.

In the case in which it is wished to obtain a greater variance it will be necessary to use additional principal components. However, the choice of the amount of principal components to use must be balanced between the variance and the degree of simplification that are wished to be obtained.

Finally, (step 260) each selected principal component has an associated input control signal so that, by varying the input control signal, the individual articulated members 11 constituting the multiarticular robotic and/or prosthetic device 10 are moved based on the relationship defined by the principal component.

The input signals can be given in the way most suitable according to the application.

For example, in the case of just two principal components it is possible to use a bi-dimensional interface like for example a mouse by means of which a bi-dimensional signal is generated depending upon the position in the plane in which the mouse is moved.

Similarly, it is also possible to use a joystick as bi-dimensional interface.

In prosthesis applications, the input signals can be of the electromyographic type.

More generally, the multiarticular robotic and/or prosthetic device 10 can be driven by means of an input signal of any kind.

The use of a small number of input signals reduces the training time needed by an operator to be able to successfully associate the inputs given by him to the movements of the device 10.

As an example the interface method between an operator and a multiarticular robotic and/or prosthetic device for moving the same shall now be illustrated based on the multiarticular device 10 illustrated in FIG. 2.

Such a device 10 has five actuators 12 respectively adapted to drive four shafts 11 hinged together and the free end of jaw gripping means 13.

The shafts 11 are driven to take up a relative angular position one with respect to the one next to it, whereas the shanks of the gripping means 13 are controlled to take up a relative position brought together or apart.

In order to determine the principal components a moving strategy of the multiarticular device 10 is initially selected.

For this purpose moving rules are also determined, like for example that the four shafts 11 must never be aligned, or else that all four of the shafts 11 can bend at most by a certain number of degrees, and so on.

Once the moving rules of the device 10 within a predetermined moving space have been defined, a discrete number of positions that the device 10 can take up within at least one portion of the moving space is defined, respecting such moving rules.

In the example case, the multiarticular device 10 can take up positions such that it can collect the objects 14 schematised in the figure by balls.

Preferably, in selecting the defined positions, it is attempted to more or less cover the entire moving space that can be reached by the device 10.

For each position defined, the angular positions of the individual shafts 11 and of the shanks of the gripping means 13 are then recorded.

Such data is then processed so as to obtain the principal components of the system based on which to carry out the interfacing between the signals given by the operator and those for driving the multiarticular device 10.

In the specific case with five degrees of freedom, five principal components will be obtained, of which the components able to offer the greater variance will be used.

The choice of how many principal components to use in the interface method according to the present invention derives from a balancing between the complexity for the operator to learn to use it and the degree of accuracy that the method must offer.

Similarly to the example shown, a prosthesis of a limb can also be driven through the interface method according to the present invention in which, as inputs, electromyographic signals are for example used.

From the description that has been made the characteristics of the method object of the present invention are clear, just as the relative advantages are also clear.

The interface method between an operator and a multiarticular robotic and/or prosthetic device according to the present invention does indeed offer a great ease of use and learning thanks to the simplified control obtained through the possibility of using a small number of inputs necessary to drive the device with respect to the number of signals required by the actuators to manage the degrees of freedom that it has. Therefore, it is possible to accurately drive an anthropomorphic robot having for example 20 degrees of freedom even just using a mouse or a joystick.

In particular, in the field of prostheses results in terms of reliability are obtained that are comparable to the invasive methods currently used to drive multiarticular devices whilst requiring only a short rehabilitation and motor training period.

Such a method can also be actuated by means of a small number of electromyographic signals that can be taken from the patient in a non-invasive manner.

Finally, it is clear that the method thus conceived can undergo numerous modifications and variants, all of which are covered by the invention.

1) Interface method (100) between an operator and a multiarticular robotic and/or prosthetic device (10) for moving the same characterized in that it comprises the steps consisting of:
receiving (110) as an input a plurality of control signals whose number is less than a number of signals required by a plurality of actuators (12) used for moving a plurality of movable members (11,13) of said multiarticular robotic and/or prosthetic device (10); and
converting said plurality of input control signals into a number of output signals corresponding to the number of said signals required by said actuators (12) by means of a plurality of principal components which characterize the movements of said device (10) within a moving space.

2) Interface method (100) between an operator and a multiarticular robotic and/or prosthetic device (10) for moving the same according to claim 1, characterized in that said principal components are determined according to the following steps:
selecting (210) a moving strategy of said multiarticular robotic and/or prosthetic device (10) to be driven by defining moving rules;
depending on the selected moving strategy, defining (220) a discrete set of working positions which can be taken up by said multiarticular robotic and/or prosthetic device (10) within at least one portion of said moving space;
for each defined working position, detecting (230) the angular positions of each mobile member (11,13) of said multiarticular robotic and/or prosthetic device (10);
determining (240) the principal components of a system formed by the plurality of angular positions which every mobile member (11,13) can take up in each position of said discrete set of working positions;
picking out (250), among said determined principal components, a subset of principal components adapted to provide a predetermined variance value;
associating (260) to each selected principal component, an input control signal.

3) Interface method (100) between an operator and a multiarticular robotic and/or prosthetic device (10) for moving the same according to claim 2, characterized in that said step of detecting (230) the angular positions of each mobile member (11,13) consists of determining a plurality of postures and/or movements of said multiarticular robotic and/or prosthetic device (10) through the experimental data acquisition or the determination of a model.

4) Interface method (100) between an operator and a multiarticular robotic and/or prosthetic device (10) for moving the same according to claim 2, characterized in that said predetermined variance value ranges from 70% to 90% and it is preferably 80%.

5) Interface method (100) between an operator and a multiarticular robotic and/or prosthetic device (10) for moving the same according to claim 2, characterized in that said principal components are determined by means of computing functions implemented in computing environments for an electronic computer.

6) Interface method (100) between an operator and a multiarticular robotic and/or prosthetic device (10) for moving the same according to claim 1, characterized in that said input control signals are given through a bi-dimensional interface.

7) Interface method (100) between an operator and a multiarticular robotic and/or prosthetic device (10) for moving the same according to claim 1, characterized in that said multiarticular device (10) is an anthropomorphic robot.

8) Interface method (100) between an operator and a multiarticular robotic and/or prosthetic device (10) for moving the same according to claim 1, characterized in that said input control signals are electromyographic signals.

9) Interface method (100) between an operator and a multiarticular robotic and/or prosthetic device (10) for moving the same according to claim 8, characterized in that said multiarticular device (10) is a prosthetic device.

10) Interface method (100) between an operator and a multiarticular robotic and/or prosthetic device (10) for moving the same according to claim 9, characterized in that said multiarticular prosthetic device (10) is a hand prosthesis.
